(19) United States
(12) Patent Application Publication
(10) Pub. No.: US 2023/0181598 A1
(43) Pub. Date: Jun. 15, 2023

(54) FORMULATIONS AND METHODS OF TREATING COVID-19 AND PREVENTING INFECTION WITH SARS-CoV-2

(71) Applicants: Patrick PRENDERGAST, Byrock (AU); Daniel SHANAHAN-PRENDERGAST, Kilmore Quay (IE)

(72) Inventors: Patrick PRENDERGAST, Byrock (AU); Daniel SHANAHAN-PRENDERGAST, Kilmore Quay (IE)

(21) Appl. No.: 18/166,771

(22) Filed: Feb. 9, 2023

Related U.S. Application Data
(63) Continuation of application No. PCT/US2021/042159, filed on Jul. 19, 2021.
(60) Provisional application No. 63/064,660, filed on Aug. 12, 2020.

(51) Int. Cl. A61K 31/569; A61K 31/4422; A61K 45/06; A61P 31/14; A61P 11/00

(52) U.S. Cl. CPC A61K 31/569; A61K 31/4422; A61K 45/06; A61P 31/14; A61P 11/00

(57) ABSTRACT

Provided is a method of treatment of COVID-19 and/or preventing infection with SARS-CoV-2 comprising: administering to an individual in need thereof a therapeutically effective dose of mifepristone, or a pharmaceutically acceptable salt and/or analogue thereof, and a therapeutically effective dose of amlodipine, or a pharmaceutically acceptable salt thereof.

This application is a bypass continuation of International Application No. PCT/US2021/042159, filed Jul. 19, 2021, which claims priority to, and the benefit of, U.S. Application No. 63/064,660, filed Aug. 12, 2020, the contents of each are incorporated by reference as if written herein in their entireties.

Coronaviruses are enveloped RNA viruses that are distributed broadly among humans, other mammals, and birds and that cause respiratory, enteric, hepatic, and neurologic diseases. Six coronavirus species are known to cause human disease. Four viruses—229E, OC43, NL63, and HKU1—are prevalent and typically cause common cold symptoms in immunocompetent individuals. The two other strains—severe acute respiratory syndrome coronavirus (SARS-CoV) and Middle East respiratory syndrome coronavirus (MERS-CoV)—are zoonotic in origin and have been linked to sometimes fatal illness. SARS-CoV was the causal agent of the severe acute respiratory syndrome outbreaks in 2002 and 2003 in Guangdong Province, China. MERS-CoV was the pathogen responsible for severe respiratory disease outbreaks in 2012 in the Middle East.

In December 2019, a previously unknown betacoronavirus was discovered through the use of unbiased sequencing in samples from patients with pneumonia. Human airway epithelial cells were used to isolate a novel coronavirus, named 2019-nCoV (now called SARS-CoV-2), which formed a Glade within the subgenus sarbecovirus, Orthocoronavirinae subfamily. Different from both MERS-CoV and SARS-CoV, SARS-CoV-2 is the seventh member of the family of coronaviruses that infect humans.

The severe respiratory illness caused by SARS-CoV-2 is now called Coronavirus Disease 2019 or COVID-19. No specific anti-viral treatment for COVID-19 exists. SARS-CoV-2 infection is a global health crisis. Lethal complications are caused by damage and failure of vital organs that express high levels of ACE2, including the lungs, heart and the kidneys. Like other betacoronaviruses SARS-Co-V2 binds to a receptor on its host cells via its outer spike (S) proteins, after which the spike protein is primed to mediate fusion of the viral cell membrane with the host cell membrane to gain viral cell entry. The host cell receptor for SARS-Co-V2 has recently been determined to be angiotensin-converting enzyme 2 (ACE2) and priming requires the activity of the transmembrane protease serine 2 (TMPRSS2) encoded by TMPRSS2 gene.

In order to gain entry into the host cell, both SARS-CoV and MERS-CoV can be triggered to fuse early at the plasma membrane or later at the endosomal membrane depending on the availability of the priming protease. The preferred pathway for MERS is the plasma membrane pathway if TMPRSS2 is available. Recent data indicates that SARS-CoV-2 can also use both fusion pathways, but if the TMPRS22 pathway is inhibited, with the specific protease inhibitor camostat mesylate, then viral entry and infection can occur via the endosomal pathway. This later pathway requires priming of the S protein by the pH dependent protease cathepsin-L. Thus, it can be hypothesized that an effective anti-viral treatment for SARS-Co-V2 should inhibit both the early TMPRSS2-dependent and the later endosomal/cathepsin-L-dependent cell entry pathways.

Ideally treatment should not only result in a reduction of the viral load but also prevent the hyperinflammation in the lungs that leads to the development of acute respiratory distress syndrome (ARDS) and ultimately, death. One of the main mechanisms for ARDS in these viral respiratory syndromes is an uncontrolled, immune-mediated, systemic inflammatory response resulting from the release of large amounts of pro-inflammatory cytokines and chemokines by immune effector cells. Known as a “cytokine storm”, this dysregulated and excessive immune response triggers a violent attack to the body, characterized by massive inflammatory cell infiltration, elevated pro-inflammatory cytokine/chemokine responses and subsequent acute lung injury (ALI). The development of ALI involves increased lung vascular permeability and alveolar flooding, resulting in reduced alveolar gas exchange and ARDS with eventual multisystem organ failure (MSOF) and death.

There exists a need for effectively treating individuals having COVID-19 or at risk of infection with SARS-CoV-2. The present disclosure satisfies this need and provides related advantages as well.

Citation of any reference throughout this application is not to be construed as an admission that such reference is prior art to the present application.

### SUMMARY

Provided is a method of treatment of COVID-19 comprising: administering daily to an individual in need thereof a therapeutically effective dose of mifepristone, or a pharmaceutically acceptable salt and/or analogue thereof, and a therapeutically effective dose of amlodipine, or a pharmaceutically acceptable salt thereof.

Also provided is a method of treatment of COVID-19 hyperinflammation comprising: administering to an individual in need thereof a therapeutically effective dose of mifepristone, or a pharmaceutically acceptable salt and/or analogue thereof, and a therapeutically effective dose of amlodipine, or a pharmaceutically acceptable salt thereof.

Also provided is a method of reducing, retarding or otherwise inhibiting growth and/or replication of SARS-CoV-2 in an individual confirmed to have COVID-19 comprising: administering to an individual in need thereof a therapeutically effective dose of mifepristone, or a pharmaceutically acceptable salt and/or analogue thereof, and a therapeutically effective dose of amlodipine, or a pharmaceutically acceptable salt thereof.

Also provided is a method of reducing lung injury resulting from a hyperaggressive immune response comprising: administering to an individual in need thereof a therapeutically effective dose of mifepristone, or a pharmaceutically acceptable salt and/or analogue thereof, and a therapeutically effective dose of amlodipine, or a pharmaceutically acceptable salt thereof.

Also provided is a method of preventing acute respiratory distress syndrome (ARDS) comprising: administering to an individual in need thereof a therapeutically effective dose of mifepristone, or a pharmaceutically acceptable salt and/or analogue thereof, and a therapeutically effective dose of amlodipine, or a pharmaceutically acceptable salt thereof.

Also provided is a method of preventing COVID-19 comprising: administering to an individual at risk of infection with SARS-CoV-2 a therapeutically effective dose of mifepristone, or a pharmaceutically acceptable and/or analogue salt thereof, and a therapeutically effective dose of, or a pharmaceutically acceptable salt thereof, prior to exposure to SARS-CoV-2.

Also provided is a method of preventing infection of an individual exposed to SARS-CoV-2 wherein the individual has not been confirmed to have COVID-19 comprising: administering to the individual a therapeutically effective dose of mifepristone, or a pharmaceutically acceptable salt and/or analogue thereof, and a therapeutically effective dose of amlodipine, or a pharmaceutically acceptable salt thereof.

Also provided is a method of blocking the entry of SARS-CoV-2 into cells via the TMPRSS2-dependent and the endosomal/cathepsin-L-dependent cell entry pathways comprising contacting the cells with a therapeutically effective dose of mifepristone, or a pharmaceutically acceptable salt and/or analogue thereof, and a therapeutically effective dose of amlodipine, or a pharmaceutically acceptable salt thereof.

Also provided is a method for maintaining vascular endothelial stability, preventing hypovolemic shock and/or improving immune cell viability in an individual infected by coronavirus comprising: administering to the individual a therapeutically effective dose of mifepristone, or a pharmaceutically acceptable salt and/or analogue thereof, and a therapeutically effective dose of amlodipine, or a pharmaceutically acceptable salt thereof.

Also provided is a method for preventing apoptosis of immune cells, particularly macrophage and dendritic cells in an individual infected with SARS-CoV-2, comprising: administering to the individual a therapeutically effective dose of mifepristone, or a pharmaceutically acceptable salt and/or analogue thereof, and a therapeutically effective dose of amlodipine, or a pharmaceutically acceptable salt thereof.

Also provided is a method for preventing vascular endothelial instability, muscle pain, and/or rheumatic pain which an individual may suffer from coronavirus infection comprising: administering to the individual a therapeutically effective dose of mifepristone, or a pharmaceutically acceptable salt and/or analogue thereof, and a therapeutically effective dose of amlodipine, or a pharmaceutically acceptable salt thereof.

These and other aspects of the invention disclosed herein will be set forth in greater detail as the patent disclosure proceeds.

### DETAILED DESCRIPTION

As used in the present specification, the following words and phrases are generally intended to have the meanings as set forth below, except to the extent that the context in which they are used indicates otherwise.

AMLODIPINE: Amlodipine is a long-acting calcium channel blocker. NORVASC is the besylate salt of amlodipine and is chemically described as 3-Ethyl-5-methyl (±)-2-[(2-aminoethoxy)methyl]4-(2-chlorophenyl)-1,4-dihydro-6-methyl-3,5-pyridinedicarboxylate, monobenzenesulphonate. Its empirical formula is C₂₆H₃₁ClN₂O₈S. Amlodipine besylate is a white crystalline powder with a molecular weight of 567.1. It is slightly soluble in water and sparingly soluble in ethanol.

Amlodipine besylate is approved for use alone or in combination with other antihypertensive and antianginal agents for the treatment of: Hypertension, Coronary Artery Disease, Chronic Stable Angina, Vasospastic Angina (Prinzmetal's or Variant Angina), Angiographically Documented Coronary Artery Disease in patients without heart failure or an ejection fraction <40%. The recommended starting dose for adults for those indications is 5 mg once daily with maximum dose 10 mg once daily. Small, fragile, or elderly patients, or patients with hepatic insufficiency may be started on 2.5 mg once daily. The pediatric starting dose is 2.5 mg to 5 mg once daily.

NORVASC tablets are formulated as white tablets equivalent to 2.5, 5, and 10 mg of amlodipine for oral administration. In addition to the active ingredient, amlodipine besylate, each tablet contains the following inactive ingredients: microcrystalline cellulose, dibasic calcium phosphate anhydrous, sodium starch glycolate, and magnesium stearate.

As used herein, amlodipine includes levamlodipine, also known as levoamlodipine or S-amlodipine, which is a pharmacologically active enantiomer of amlodipine.

MIFEPRISTONE: Mifepristone is a cortisol receptor blocker for oral administration and is chemically described as 11β-(4-dimethylaminophenyl)-17β-hydroxy-17α-(1-propynyl)-estra-4,9-dien-3-one. The chemical formula is C₂₉H₃₅NO₂; the molecular weight is 429.60. It is marketed as KORLYM® mifepristone 300 mg tablets. KORLYM is indicated to control hyperglycemia secondary to hypercortisolism in adult patients with endogenous Cushing's syndrome who have type 2 diabetes mellitus or glucose intolerance and have failed surgery or are not candidates for surgery.

Each KORLYM tablet for oral use contains 300 mg of mifepristone. The inactive ingredients of KORLYM tablets are silicified microcrystalline cellulose, sodium starch glycolate, hydroxypropylcellulose, sodium lauryl sulfate, magnesium stearate, hypromellose, titanium dioxide, triacetin, D&C yellow 10 aluminum lake, polysorbate 80, and FD&C yellow 6 aluminum lake.

Mifepristone is also marketed as MIFEPREX® 200 mg tablets for the medical termination of intrauterine pregnancy through 49 days' pregnancy. Each MIFEPREX tablet contains 200 mg of mifepristone. The tablets include the inactive ingredients colloidal silica anhydrous, corn starch, povidone, microcrystalline cellulose, and magnesium stearate.

Mifepristone demonstrates a pH-related solubility profile. The greatest solubility is achieved in acidic media (˜25 mg/mL at pH 1.5) and solubility declines rapidly as the pH is increased. At pH values above 2.5 the solubility of mifepristone is less than 1 mg/mL.

Metabolites of mifepristone that are thought to have pharmacological activity include RU42633 (desmethylmifepristone: (8S,11R,13S,14S,17S)-17-hydroxy-13-methyl-11-[4-(methylamino)phenyl]-17-prop-1-ynyl-1,2,6,7,8,11,12,14,15,16-decahydrocyclopenta[a]phenanthren-3-one); RU42698 (22-hydroxy mifepristone: (8S,11R,13S,14S,17S)-11-[4-(dimethylamino)phenyl]-17-hydroxy-17-(3-hydroxyprop-1-ynyl)-13-methyl-1,2,6,7,8,11,12,14,15,16-decahydrocyclopenta[a]phenanthren-3-one); and RU42848 (didesmethylmifepristone: (8S,11R,13S,14S,17S)-11-(4-aminophenyl)-17-hydroxy-13-methyl-17-prop-1-ynyl-1,2,6,7,8,11,12,14,15,16-decahydrocyclopenta[a]phenanthren-3-one).

Analogs of mifepristone have been described. See, e.g., DeBono, A., Thomas, D. R., Lundberg, L. et al. Novel RU486 (mifepristone) analogues with increased activity against Venezuelan Equine Encephalitis Virus but reduced progesterone receptor antagonistic activity. Sci Rep 9, 2634 (2019), which is incorporated herein by reference for all purposes. In some embodiments, mifepristone refers to an analog of mifepristone. In some embodiments, the mifepristone analogue is PT150 (aka Organon 34517) which has the structure

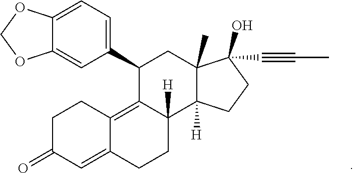

MODERATE SEVERITY COVID-19: As used herein, moderate severity COVID-19 refers to: individuals who have evidence of lower respiratory disease by clinical assessment or imaging and a saturation of oxygen (SpO₂)≥94% on room air at sea level. While the diagnosis can be made on clinical grounds; chest imaging (radiograph, CT scan, ultrasound) may assist in diagnosis and identify or exclude pulmonary complications.

STANDARD OF CARE: As used herein, “standard of care” or “SOC” refers to the diagnostic and treatment process that a clinician should follow for a certain type of patient, illness, or clinical circumstance. SOC may include administration of drugs that are being used in clinical practice for the treatment of COVID-19 (e.g. lopinavir/ritonavir; darunavir/cobicistat; hydroxy/chloroquine, tocilizumab, etc.), other than those used as part of another clinical trial.

ACUTE RESPIRATORY DISTRESS SYNDROME OR ARDS: As used herein, acute respiratory distress syndrome or ARDS is a type of respiratory failure characterized by rapid onset of widespread inflammation in the lungs. Symptoms include shortness of breath, rapid breathing, and bluish skin coloration.

ADMINISTERING: As used herein, “administering” means to provide a compound or other therapy, remedy, or treatment such that an individual internalizes a compound.

PRESCRIBING: As used herein, “prescribing” means to order, authorize, or recommend the use of a drug or other therapy, remedy, or treatment. In some embodiments, a health care practitioner can orally advise, recommend, or authorize the use of a compound, dosage regimen or other treatment to an individual. In this case the health care practitioner may or may not provide a prescription for the compound, dosage regimen, or treatment. Further, the health care practitioner may or may not provide the recommended compound or treatment. For example, the health care practitioner can advise the individual where to obtain the compound without providing the compound. In some embodiments, a health care practitioner can provide a prescription for the compound, dosage regimen, or treatment to the individual. For example, a health care practitioner can give a written or oral prescription to an individual. A prescription can be written on paper or on electronic media such as a computer file, for example, on a hand-held computer device. For example, a health care practitioner can transform a piece of paper or electronic media with a prescription for a compound, dosage regimen, or treatment. In addition, a prescription can be called in (oral), faxed in (written), or submitted electronically via the internet to a pharmacy or a dispensary. In some embodiments, a sample of the compound or treatment can be given to the individual. As used herein, giving a sample of a compound constitutes an implicit prescription for the compound. Different health care systems around the world use different methods for prescribing and/or administering compounds or treatments and these methods are encompassed by the disclosure.

A prescription can include, for example, an individual's name and/or identifying information such as date of birth. In addition, for example, a prescription can include: the medication name, medication strength, dose, frequency of administration, route of administration, number or amount to be dispensed, number of refills, physician name, physician signature, and the like. Further, for example, a prescription can include a DEA number and/or state number.

A healthcare practitioner can include, for example, a physician, nurse, nurse practitioner, or other related health care professional who can prescribe or administer compounds (drugs) for the treatment of a disease or disorder. In addition, a healthcare practitioner can include anyone who can recommend, prescribe, administer, or prevent an individual from receiving a compound or drug including, for example, an insurance provider.

PREVENT, PREVENTING, OR PREVENTION: As used herein, the term “prevent,” “preventing”, or “prevention” disease or disorder or the occurrence or onset of one or more symptoms associated with the particular disorder and does not necessarily mean the complete prevention of the disorder. For example, the term “prevent,” “preventing” and “prevention” means the administration of therapy on a prophylactic or preventative basis to an individual who may ultimately manifest at least one symptom of a disease or condition but who has not yet done so. Such individuals can be identified on the basis of risk factors that are known to correlate with the subsequent occurrence of the disease. Alternatively, prevention therapy can be administered without prior identification of a risk factor, as a prophylactic measure. Delaying the onset of at least one symptom can also be considered prevention or prophylaxis.

TREAT, TREATING, OR TREATMENT: As used herein the term “treat,” “treating”, or “treatment” means the administration of therapy to an individual who already manifests at least one symptom of a disease or condition or who has previously manifested at least one symptom of a disease or condition. For example, “treating” can include alleviating, abating or ameliorating a disease or one or more condition symptoms, preventing one or more additional symptoms, ameliorating the underlying metabolic causes of one or more symptoms, inhibiting the disease or condition, e.g., arresting the development of the disease or condition, relieving the disease or condition, causing regression of the disease or condition, relieving a condition caused by the disease or condition, or stopping one or more of the symptoms of the disease or condition. For example, the term “treating” in reference to a disorder means a reduction in severity of one or more symptoms associated with that particular disorder. Therefore, treating a disorder does not necessarily mean a reduction in severity of all symptoms associated with a disorder and does not necessarily mean a complete reduction in the severity of one or more symptoms associated with a disorder.

TOLERATE: As used herein, an individual is said to “tolerate” a dose of a compound if administration of that dose to that individual does not result in an unacceptable adverse event or an unacceptable combination of adverse events. One of skill in the art will appreciate that tolerance is a subjective measure and that what may be tolerable to one individual may not be tolerable to a different individual. For example, one individual may not be able to tolerate headache, whereas a second individual may find headache tolerable but is not able to tolerate vomiting, whereas for a third individual, either headache alone or vomiting alone is tolerable, but the individual is not able to tolerate the combination of headache and vomiting, even if the severity of each is less than when experienced alone.

ADVERSE EVENT: As used herein, an “adverse event” is an untoward medical occurrence that is associated with treatment. Adverse events associated with mifepristone include nausea, fatigue, headache, decreased blood potassium, arthralgia, vomiting, peripheral edema, hypertension, dizziness, decreased appetite, endometrial hypertrophy. Adverse events associated with amlodipine besylate include headache and edema which occurred in a dose related manner. Other adverse experiences not dose related but reported with an incidence >1.0% are headache, fatigue, nausea, abdominal pain, and somnolence.

IN NEED OF TREATMENT and IN NEED THEREOF: As used herein, “in need of treatment” and “in need thereof” when referring to treatment are used interchangeably to mean a judgment made by a caregiver (e.g. physician, nurse, nurse practitioner, etc. in the case of humans) that an individual requires or will benefit from treatment. This judgment is made based on a variety of factors that are in the realm of a caregiver's expertise, but that includes the knowledge that the individual is ill, or will become ill, as the result of a disease, condition or disorder that is treatable by the compounds of the invention. Accordingly, the compounds of the invention can be used in a protective or preventive manner; or compounds of the invention can be used to alleviate, inhibit or ameliorate the disease, condition or disorder.

OUTPATIENT: As used herein, an “outpatient” is an individual ho receives medical treatment without being admitted to a hospital.

DOSE: As used herein, “dose” means a quantity of a pharmaceutical agent given to the individual for treating or preventing the disease or disorder at one specific time.

LOADING DOSE: As used herein “loading dose” means an initial quantity of a pharmaceutical agent, given to an individual for treating or preventing the disease or disorder at one specific time, that is higher than the dose for subsequent days of treatment.

PHARMACEUTICAL COMPOSITION: As used herein, “pharmaceutical composition” means a composition comprising at least one active ingredient whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

Alternatively, the dosage amounts disclosed herein can be replaced with dosage amounts for other salt or crystalline forms, formulations, and dosage regimens that exhibit bioequivalence to the specified amount of free acid or base, including forms with 80-125% of the AUC and/or Cₘₐₓ for the specified amount of free acid or base as measured by method disclosed in the FDA's Guidance for Industry for Bioavailability and Bioequivalence (Guidance for Industry: Bioavailability and Bioequivalence Studies for Orally Administered Drug Products—General Considerations. U.S. Department of Health and Human Services, Food and Drug Administration, Center for Drug Evaluation and Research (CDER), March 2003, Revision 1, www.fda.gov/cder/guidance/index.htm).

The compounds according to the invention may optionally exist as pharmaceutically acceptable salts including pharmaceutically acceptable acid addition salts prepared from pharmaceutically acceptable non-toxic acids including inorganic and organic acids. Representative acids include, but are not limited to, acetic, benzenesulfonic, benzoic, camphorsulfonic, citric, ethenesulfonic, dichloroacetic, formic, fumaric, gluconic, glutamic, hippuric, hydrobromic, hydrochloric, isethionic, lactic, maleic, malic, mandelic, methanesulfonic, mucic, nitric, oxalic, pamoic, pantothenic, phosphoric, succinic, sulfiric, tartaric, oxalic, p-toluenesulfonic and the like, such as those pharmaceutically acceptable salts listed by Berge et al., Journal of Pharmaceutical Sciences, 66:1-19 (1977), incorporated herein by reference in its entirety.

The acid addition salts may be obtained as the direct products of compound synthesis. In the alternative, the free base may be dissolved in a suitable solvent containing the appropriate acid and the salt isolated by evaporating the solvent or otherwise separating the salt and solvent. The compounds of this invention may form solvates with standard low molecular weight solvents using methods known to the skilled artisan.

When an integer is used in a method disclosed herein, the term “about” can be inserted before the integer.

Throughout this specification, unless the context requires otherwise, the word “comprise”, or variations such as “comprises” or “comprising” will be understood to imply the inclusion of a stated step or element or integer or group of steps or elements or integers but not the exclusion of any other step or element or integer or group of elements or integers.

Throughout this specification, unless specifically stated otherwise or the context requires otherwise, reference to a single step, composition of matter, group of steps, or group of compositions of matter shall be taken to encompass one and a plurality (i.e. one or more) of those steps, compositions of matter, groups of steps, or groups of compositions of matter.

Each embodiment described herein is to be applied mutatis mutandis to each and every other embodiment unless specifically stated otherwise.

Those skilled in the art will appreciate that the invention(s) described herein is susceptible to variations and modifications other than those specifically described. It is to be understood that the invention(s) includes all such variations and modifications. The invention(s) also includes all of the steps, features, compositions and compounds referred to or indicated in this specification, individually or collectively, and any and all combinations or any two or more of said steps or features unless specifically stated otherwise.

The present invention(s) is not to be limited in scope by the specific embodiments described herein, which are intended for the purpose of exemplification only. Functionally-equivalent products, compositions, and methods are clearly within the scope of the invention(s), as described herein.

It is appreciated that certain features of the invention(s), which are, for clarity, described in the context of separate embodiments, can also be provided in combination in a single embodiment. Conversely, various features of the invention(s), which are, for brevity, described in the context of a single embodiment, can also be provided separately or in any suitable subcombination.

Provided is a method of treatment of COVID-19 comprising: administering daily to an individual in need thereof a therapeutically effective dose of mifepristone, or a pharmaceutically acceptable salt and/or analogue thereof, and a therapeutically effective dose of amlodipine, or a pharmaceutically acceptable salt thereof.

Also provided is a method of treatment of COVID-19 hyperinflammation comprising: administering to an individual in need thereof a therapeutically effective dose of mifepristone, or a pharmaceutically acceptable salt and/or analogue thereof, and a therapeutically effective dose of amlodipine, or a pharmaceutically acceptable salt thereof.

Also provided is a method of reducing, retarding or otherwise inhibiting growth and/or replication of SARS-CoV-2 in an individual confirmed to have COVID-19 comprising: administering to an individual in need thereof a therapeutically effective dose of mifepristone, or a pharmaceutically acceptable salt and/or analogue thereof, and a therapeutically effective dose of amlodipine, or a pharmaceutically acceptable salt thereof.

Also provided is a method of reducing lung injury resulting from a hyperaggressive immune response comprising: administering to an individual in need thereof a therapeutically effective dose of mifepristone, or a pharmaceutically acceptable salt and/or analogue thereof, and a therapeutically effective dose of amlodipine, or a pharmaceutically acceptable salt thereof.

Also provided is a method of preventing acute respiratory distress syndrome (ARDS) comprising: administering to an individual in need thereof a therapeutically effective dose of mifepristone, or a pharmaceutically acceptable salt and/or analogue thereof, and a therapeutically effective dose of amlodipine, or a pharmaceutically acceptable salt thereof.

Also provided is a method of preventing COVID-19 comprising: administering to an individual at risk of infection with SARS-CoV-2 a therapeutically effective dose of mifepristone, or a pharmaceutically acceptable salt and/or analogue thereof, and a therapeutically effective dose of, or a pharmaceutically acceptable salt thereof, prior to exposure to SARS-CoV-2.

Also provided is a method of preventing infection of an individual exposed to SARS-CoV-2 wherein the individual has not been confirmed to have COVID-19 comprising: administering to the individual a therapeutically effective dose of mifepristone, or a pharmaceutically acceptable salt and/or analogue thereof, and a therapeutically effective dose of amlodipine, or a pharmaceutically acceptable salt thereof.

Also provided is a method of blocking the of SARS-CoV-2 into cells via the TMPRSS2-dependent and the endosomal/cathepsin-L-dependent cell entry pathways comprising contacting the cells with a therapeutically effective dose of mifepristone, or a pharmaceutically acceptable salt and/or analogue thereof, and a therapeutically effective dose of amlodipine, or a pharmaceutically acceptable salt thereof.

Also provided is a method for maintaining vascular endothelial stability, preventing hypovolemic shock and/or improving immune cell viability in an individual infected by coronavirus comprising: administering to the individual a therapeutically effective dose of mifepristone, or a pharmaceutically acceptable salt and/or analogue thereof, and a therapeutically effective dose of amlodipine, or a pharmaceutically acceptable salt thereof.

Also provided is a method for preventing apoptosis of immune cells, particularly macrophage and dendritic cells in an individual infected with SARS-CoV-2, comprising: administering to the individual a therapeutically effective dose of mifepristone, or a pharmaceutically acceptable salt and/or analogue thereof, and a therapeutically effective dose of amlodipine, or a pharmaceutically acceptable salt thereof.

Also provided is a method for preventing vascular endothelial instability, muscle pain, and/or rheumatic pain which an individual may suffer from coronavirus infection comprising: administering to the individual a therapeutically effective dose of mifepristone, or a pharmaceutically acceptable salt and/or analogue thereof, and a therapeutically effective dose of amlodipine, or a pharmaceutically acceptable salt thereof.

In some embodiments, the COVID-19 is of moderate severity.

In some embodiments, the administration of mifepristone, or a pharmaceutically acceptable salt and/or analogue thereof, and the amlodipine, or a pharmaceutically acceptable salt thereof, is conducted on an outpatient basis.

In some embodiments, the administration of mifepristone, or a pharmaceutically acceptable salt and/or analogue thereof, and the amlodipine, or a pharmaceutically acceptable salt thereof, reduces viral load and prevents disease progression and hospitalization.

In some embodiments, the administration of mifepristone, or a pharmaceutically acceptable salt and/or analogue thereof, and the amlodipine, or a pharmaceutically acceptable salt thereof, inhibits the release of pro-inflammatory cytokines and chemokines by immune effector cells.

In some embodiments, the administration of mifepristone, or a pharmaceutically acceptable salt and/or analogue thereof, and the amlodipine, or a pharmaceutically acceptable salt thereof, provides a therapeutically effective plasma concentration or a therapeutically effective concentration at the site of infection of mifepristone, or a pharmaceutically acceptable salt and/or analogue thereof, and a therapeutically effective plasma concentration or a therapeutically effective concentration at the site of infection of the amlodipine, or a pharmaceutically acceptable salt thereof.

In some embodiments, the administration provides a therapeutically effective plasma concentration of mifepristone, or a pharmaceutically acceptable salt and/or analogue thereof, and a therapeutically effective plasma concentration of the amlodipine, or a pharmaceutically acceptable salt thereof.

In some embodiments, the administration of mifepristone, or a pharmaceutically acceptable salt and/or analogue thereof, and the amlodipine, or a pharmaceutically acceptable salt thereof, provides a therapeutically effective concentration at the site of infection of mifepristone, or a pharmaceutically acceptable salt and/or analogue thereof, and a therapeutically effective concentration at the site of infection of the amlodipine, or a pharmaceutically acceptable salt thereof.

In some embodiments, the therapeutically effective plasma concentration of mifepristone and/or its metabolites, or a pharmaceutically acceptable salt and/or analogue of mifepristone and/or its metabolites thereof is 500 ng/mL to 12,000 ng/mL.

In some embodiments, the therapeutically effective plasma concentration of mifepristone and/or its metabolites, or a pharmaceutically acceptable salt and/or analogue of mifepristone and/or its metabolites thereof is 500 ng/mL to 10,000 ng/mL.

In some embodiments, the therapeutically effective plasma concentration of amlodipine, or a pharmaceutically acceptable salt and/or analogue thereof is 0.1 to 10 ng/mL.

In some embodiments, the therapeutically effective plasma concentration of amlodipine, or a pharmaceutically acceptable salt and/or analogue thereof is 0.5 to 10 ng/mL.

In some embodiments, the therapeutically effective dose of mifepristone, or a pharmaceutically acceptable salt and/or analogue thereof is at least about 300 mg daily. In some embodiments, the therapeutically effective dose of mifepristone, or a pharmaceutically acceptable salt and/or analogue thereof is about 300 mg daily. In some embodiments, the therapeutically effective dose of mifepristone, or a pharmaceutically acceptable salt and/or analogue thereof is at least about 600 mg daily. In some embodiments, the therapeutically effective dose of mifepristone, or a pharmaceutically acceptable salt and/or analogue thereof is about 600 mg daily. In some embodiments, the therapeutically effective dose of mifepristone, or a pharmaceutically acceptable salt and/or analogue thereof is at least about 900 mg daily. In some embodiments, the therapeutically effective dose of mifepristone, or a pharmaceutically acceptable salt and/or analogue thereof is about 900 mg daily. In some embodiments, the therapeutically effective dose of mifepristone, or a pharmaceutically acceptable salt and/or analogue thereof is at least about 1200 mg daily. In some embodiments, the therapeutically effective dose of mifepristone, or a pharmaceutically acceptable salt and/or analogue thereof is about 1200 mg daily.

In some embodiments, the therapeutically effective dose of mifepristone, or a pharmaceutically acceptable salt and/or analogue thereof is at least about 200 mg daily. In some embodiments, the therapeutically effective dose of mifepristone, or a pharmaceutically acceptable salt and/or analogue thereof is about 200 mg daily. In some embodiments, the therapeutically effective dose of mifepristone, or a pharmaceutically acceptable salt and/or analogue thereof is at least about 400 mg daily. In some embodiments, the therapeutically effective dose of mifepristone, or a pharmaceutically acceptable salt and/or analogue thereof is about 400 mg daily. In some embodiments, the therapeutically effective dose of mifepristone, or a pharmaceutically acceptable salt and/or analogue thereof is at least about 800 mg daily. In some embodiments, the therapeutically effective dose of mifepristone, or a pharmaceutically acceptable salt and/or analogue thereof is about 800 mg daily. In some embodiments, the therapeutically effective dose of mifepristone, or a pharmaceutically acceptable salt and/or analogue thereof is at least about 1000 mg daily. In some embodiments, the therapeutically effective dose of mifepristone, or a pharmaceutically acceptable salt and/or analogue thereof is about 1000 mg daily.

In some embodiments, the therapeutically effective dose of amlodipine, or a pharmaceutically acceptable salt thereof is at least about 5 mg daily. In some embodiments, the therapeutically effective dose of amlodipine, or a pharmaceutically acceptable salt thereof is at least about 10 mg daily. In some embodiments, the therapeutically effective dose of amlodipine, or a pharmaceutically acceptable salt thereof is about 10 mg daily.

In some embodiments, the amlodipine, or a pharmaceutically acceptable salt thereof, is amlodipine besylate. In some embodiments, the amlodipine, or a pharmaceutically acceptable salt thereof, is amlodipine maleate.

In some embodiments, the amlodipine, or a pharmaceutically acceptable salt thereof, is levamlodipine, or a pharmaceutically acceptable salt thereof. In some embodiments, the amlodipine, or a pharmaceutically acceptable salt thereof, is levamlodipine maleate. In some embodiments, the amlodipine, or a pharmaceutically acceptable salt thereof, is levamlodipine besylate.

In some embodiments, the therapeutically effective dose of mifepristone, or a pharmaceutically acceptable salt and/or analogue thereof, and the therapeutically effective dose of amlodipine, or a pharmaceutically acceptable salt thereof is administered for 2 to 20 or more days. In some embodiments, the therapeutically effective dose of mifepristone, or a pharmaceutically acceptable salt and/or analogue thereof, and the therapeutically effective dose of amlodipine, or a pharmaceutically acceptable salt thereof is administered for 2 to 14 days. In some embodiments, the therapeutically effective dose of mifepristone, or a pharmaceutically acceptable salt and/or analogue thereof, and the therapeutically effective dose of amlodipine, or a pharmaceutically acceptable salt thereof is administered for 3 to 12 days. In some embodiments, the therapeutically effective dose of mifepristone, or a pharmaceutically acceptable salt and/or analogue thereof, and the therapeutically effective dose of amlodipine, or a pharmaceutically acceptable salt thereof is administered for 5 to 10 days.

In some embodiments, the therapeutically effective dose of mifepristone, or a pharmaceutically acceptable salt and/or analogue thereof, and the therapeutically effective dose of amlodipine, or a pharmaceutically acceptable salt thereof is administered to the individual when the individual is in the fasted state.

In some embodiments, the therapeutically effective dose of mifepristone, or a pharmaceutically acceptable salt and/or analogue thereof, and the therapeutically effective dose of amlodipine, or a pharmaceutically acceptable salt thereof is administered to the individual when the individual is in the fed state.

In some embodiments, administration is initiated within the earlier of 24 to 72 hours of illness onset or confirmation of the individual having COVID-19.

In some embodiments, administration is initiated within the earlier of 24 hours of illness onset or confirmation of the individual having COVID-19.

In some embodiments, the individual is at an elevated risk of exposure to SARS-CoV-2. In some embodiments, the individual is a health care worker. In some embodiments, the individual is located in an area where ongoing community spread of SARS-CoV-2 has been reported. In some embodiments, the individual has been in close contacts with one or more persons with COVID-19.

In some embodiments, the individual is at an elevated risk of severe illness.

In some embodiments, the individual is 60 years of age or older.

In some embodiments, the individual has a serious chronic medical condition.

In some embodiments, the chronic medical condition is chosen from pulmonary disease, diabetes mellitus (type 2), requiring oral medication or insulin for treatment, hypertension, cardiovascular disease.

In some embodiments, the individual has a baseline blood pressure under 110 mmHg systolic at rest.

In some embodiments, the individual has a body mass index≥30.

In some embodiments, the method further comprises testing the individual for SARS-CoV-2 infection. In some embodiments, testing comprises testing nasopharyngeal and oropharyngeal swabs by real-time reverse-transcriptase-polymerase-chain-reaction (rRT-PCR) assay. A description of this assay and sequence information for the rRT-PCR panel primers and probes are available on the CDC Laboratory Information website for 2019-nCoV, which is incorporated herein by reference in its entirety.

In some embodiments, the method further comprises monitoring for adverse events during the administration.

In some embodiments, if the individual is a woman of reproductive potential, the method further comprises determining whether the individual is pregnant. In some embodiments, in the individual is a woman of reproductive potential, the method further comprises obtaining a negative pregnancy test prior to initiating administration.

In some embodiments, the method further comprises administering an antiviral drug. In some embodiments, the antiviral drug is selected from oseltamivir, remdesivir, an interferon, camostat mesylate, nafamostat mesylate, hydroxychloroquine, chloroquine, ivermectin, ribavirin, adefovir, tenofovir, acyclovir, brivudin, cidofovir, fomivirsen, foscarnet, ganciclovir, penciclovir, amantadine, rimantadine and zanamivir.

In some embodiments the individual is administered a loading dose of mifepristone, or a pharmaceutically acceptable salt and/or analogue thereof, and the amlodipine, or a pharmaceutically acceptable salt thereof, on the first day of administration or the first and second days of administration. In some embodiments, the individual is administered the same daily dose of the mifepristone, or a pharmaceutically acceptable salt and/or analogue thereof, and the amlodipine, or a pharmaceutically acceptable salt thereof, on the first and second days of administration. In some embodiments, the individual is administered the same daily dose of the mifepristone, or a pharmaceutically acceptable salt and/or analogue thereof, and the amlodipine, or a pharmaceutically acceptable salt thereof on at least each of the first 2, 3, 4, 5, 6, or 7 days of administration. In some embodiments, the individual is administered the same daily dose of the mifepristone, or a pharmaceutically acceptable salt and/or analogue thereof, and the amlodipine, or a pharmaceutically acceptable salt thereof, throughout treatment. In some embodiments, the mifepristone, or a pharmaceutically acceptable salt and/or analogue thereof, and the amlodipine, or a pharmaceutically acceptable salt is not titrated.

In some embodiments, the mifepristone, or a pharmaceutically acceptable salt and/or analogue thereof, and/or amlodipine, or the pharmaceutically acceptable salt thereof is administered orally. In some embodiments, the mifepristone, or a pharmaceutically acceptable salt and/or analogue thereof, and the amlodipine, or a pharmaceutically acceptable salt thereof is administered orally.

In some embodiments, the mifepristone, or a pharmaceutically acceptable salt and/or analogue thereof, and/or amlodipine, or the pharmaceutically acceptable salt thereof are administered at about the same time. In some embodiments, the mifepristone, or a pharmaceutically acceptable salt and/or analogue thereof, and/or amlodipine, or the pharmaceutically acceptable salt thereof are administered at different times.

In some embodiments, the mifepristone, or a pharmaceutically acceptable salt and/or analogue thereof, and/or the amlodipine, or a pharmaceutically acceptable salt thereof, is formulated as a capsule or tablet suitable for oral administration.

In some embodiments, the method further comprises monitoring for adverse events during the administration of the mifepristone, or a pharmaceutically acceptable salt and/or analogue thereof, and the amlodipine, or a pharmaceutically acceptable salt thereof optionally, interrupting or terminating the administration.

In some embodiments, the method further comprises monitoring for adverse events during the administration of the mifepristone, or a pharmaceutically acceptable salt and/or analogue thereof, the amlodipine, or the pharmaceutically acceptable salt thereof.

Also provided are pharmaceutical compositions comprising a therapeutically effective dose of the mifepristone, or a pharmaceutically acceptable salt and/or analogue thereof, and the therapeutically effective dose of amlodipine, or a pharmaceutically acceptable salt thereof and, optionally, one or more pharmaceutically acceptable carriers. Also provided are pharmaceutical compositions comprising the mifepristone, or a pharmaceutically acceptable salt and/or analogue thereof, and the amlodipine, or a pharmaceutically acceptable salt thereof, optionally, one or more pharmaceutically acceptable carriers. The carrier(s) must be “acceptable” in the sense of being compatible with the other ingredients of the formulation and not overly deleterious to the recipient thereof.

Proper formulation is dependent upon the route of administration chosen. Any of the well-known techniques, carriers, and excipients may be used as suitable and as understood in the art. The pharmaceutical compositions disclosed herein may be manufactured in any manner known in the art, e.g., by means of conventional mixing, dissolving, granulating, dragee-making, levigating, emulsifying, encapsulating, entrapping or compression processes.

The formulations include those suitable for oral, inhalation (oral or intranasal), parenteral (including subcutaneous, intradermal, intramuscular, intravenous, intraarticular, and intramedullary), intraperitoneal, transmucosal, transdermal, rectal and topical (including dermal, buccal, sublingual and intraocular) administration although the most suitable route may depend upon for example the condition and disorder of the recipient. The formulations may conveniently be presented in unit dosage form and may be prepared by any of the methods well known in the art of pharmacy. Typically, these methods include the step of bringing into association a compound of the subject disclosure or a pharmaceutically acceptable salt, ester, amide, prodrug or solvate thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

In some embodiments, the mifepristone, or a pharmaceutically acceptable salt and/or analogue thereof, and the amlodipine, or a pharmaceutically acceptable salt thereof, is administered as a raw or pure chemical, for example as a powder in capsule formulation.

Amlodipine, or a pharmaceutically acceptable salt thereof, is formulated as a pharmaceutical composition further comprising one or more pharmaceutically acceptable carriers.

Pharmaceutical compositions may be prepared by any suitable method, typically by uniformly mixing the active compound(s) with liquids or finely divided solid carriers, or both, in the required proportions and then, if necessary, forming the resulting mixture into a desired shape.

Conventional excipients, such as binding agents, fillers, acceptable wetting agents, tabletting lubricants and disintegrants may be used in tablets and capsules for oral administration. The compounds described herein can be formulated into pharmaceutical compositions using techniques well known to those in the art. Suitable pharmaceutically acceptable carriers, outside those mentioned herein, are known in the art; for example, see Remington, The Science and Practice of Pharmacy, 20^{th} Edition, 2000, Lippincott Williams & Wilkins, (Editors: Gennaro et al.)

For oral administration, the pharmaceutical composition may be in the form of, for example, a tablet or capsule. The pharmaceutical composition is preferably made in the form of a dosage unit containing a particular amount of the active ingredient. Examples of such dosage units are capsules, tablets, powders, granules or suspensions, with conventional additives such as lactose, mannitol, corn starch or potato starch; with binders such as crystalline cellulose, cellulose derivatives, acacia, corn starch or gelatins; with disintegrators such as corn starch, potato starch or sodium carboxymethyl-cellulose; and with lubricants such as talc or magnesium stearate. Solid form preparations include powders, tablets, pills, capsules, cachets, suppositories, and dispersible granules. A solid carrier can be one or more substances which may also act as diluents, flavoring agents, solubilizers, lubricants, suspending agents, binders, preservatives, tablet disintegrating agents, or encapsulating materials. In some embodiments, the pharmaceutical composition further comprises one or more surfactants.

In powders, the carrier is a finely divided solid which is in a mixture with the finely divided active component.

In tablets, the active component is mixed with the carrier having the necessary binding capacity in suitable proportions and compacted to the desired shape and size.

The powders and tablets may contain varying percentage amounts of the active compound. A representative amount in a powder or tablet may be from 0.5 to about 90 percent of the active compound. However, an artisan would know when amounts outside of this range are necessary. Suitable carriers for powders and tablets include magnesium carbonate, magnesium stearate, talc, sugar, lactose, pectin, dextrin, starch, gelatin, tragacanth, methylcellulose, sodium carboxymethyl cellulose, a low melting wax, cocoa butter, and the like. The term “preparation” includes the formulation of the active compound with encapsulating material as carrier providing a capsule in which the active component, with or without carriers, is surrounded by a carrier, which is thus in association with it. Similarly, cachets and lozenges are included. Tablets, powders, capsules, pills, cachets, and lozenges can be used as solid forms suitable for oral administration.

Compounds of the present disclosure may be administered directly into the blood stream, muscle, or internal organs by injection, e.g., by bolus injection or continuous infusion. Suitable means for parenteral administration include intravenous, intra-muscular, subcutaneous intraarterial, intraperitoneal, intrathecal, intracranial, and the like. Suitable devices for parenteral administration include injectors (including needle and needle-free injectors) and infusion methods. The formulations may be presented in unit-dose or multi-dose containers, for example sealed ampoules and vials.

Most parenteral formulations are aqueous solutions containing excipients, including salts, buffering, suspending, stabilizing and/or dispersing agents, antioxidants, bacteriostats, preservatives, and solutes which render the formulation isotonic with the blood of the intended recipient, and carbohydrates.

Parenteral formulations may also be prepared in a dehydrated form (e.g., by lyophilization) or as sterile non-aqueous solutions. These formulations can be used with a suitable vehicle, such as sterile water. Solubility-enhancing agents may also be used in preparation of parenteral solutions. Compositions for parenteral administration may be formulated as immediate or modified release, including delayed or sustained release. Compounds may also be formulated as depot preparations. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Formulations for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

Suppositories for rectal administration of the compounds of the present disclosure can be prepared by mixing the active agent with a suitable non-irritating excipient such as cocoa butter, synthetic mono-, di-, or triglycerides, fatty acids, or polyethylene glycols which are solid at ordinary temperatures but liquid at the rectal temperature, and which will therefore melt in the rectum and release the drug.

For buccal or sublingual administration, the compositions may take the form of tablets, lozenges, pastilles, films or gels formulated in conventional manner. Such compositions may comprise the active ingredient in a flavored basis such as sucrose and acacia or tragacanth.

The compounds may also be formulated in rectal compositions such as suppositories or retention enemas, e.g., containing conventional suppository bases such as cocoa butter, polyethylene glycol, or other glycerides.

For administration by inhalation (orally or intranasally), compounds may be conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the compounds according to the disclosure may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

Other carrier materials and modes of administration known in the pharmaceutical art may also be used. Pharmaceutical compositions of the disclosure may be prepared by any of the well-known techniques of pharmacy, such as effective formulation and administration procedures. Preferred unit dosage formulations are those containing an effective dose, as herein below recited, or an appropriate fraction thereof, of the active ingredient.

It should be understood that in addition to the ingredients particularly mentioned above, the formulations described above may include other agents conventional in the art having regard to the type of formulation in question, for example those suitable for oral administration may include flavoring agents.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets or capsules. Also, the unit dosage form can be a capsule or tablet itself, or it can be the appropriate number of any of these in packaged form.

Further embodiments include the embodiments disclosed in the following Examples, which is not to be construed as limiting in any way.

### EXAMPLES

### Example 1

A phase 2, randomized, controlled study evaluating the safety and efficacy of mifepristone in combination with amlodipine in addition to standard of care (SOC) compared to SOC for the outpatient treatment of moderate severity COVID-19 will be conducted.

Patients with polymerase chain reaction (PCR) confirmed SARS-CoV-2 and diagnosis of moderate severity COVID-19 will be enrolled. This will be an outpatient study with patients receiving daily nursing visits at home during a 5-day treatment period to assess safety and efficacy of the treatments. Patients will be followed for 7 days after the end of treatment with nurse visits (Days 6 & 7) and daily phone calls on Days 8-14 and on Day 28 after the last dose.

Patients will be administered up to 1200 mg/day Mifepristone and 10 mg/day Amlodipine once daily after a meal at approximately the same time plus SOC for 5 days.

The primary objective of the study is to evaluate the change from baseline to Day 3 after randomization in nasopharyngeal SARS-CoV-2 viral load (as assessed by quantitative PCR performed in triplicate) for the two treatment groups.

Secondary objectives are:
To evaluate the change from baseline to Day 5 after randomization in nasopharyngeal SARS-CoV-2 viral load (as assessed by quantitative PCR performed in triplicate for each subject) following the two treatments
To evaluate time to clinical worsening during the 28 days following randomization. Clinical worsening is defined as meeting (1) presence of dyspnea and/or hospitalization for shortness of breath or pneumonia, plus at least one of the following: (2) decrease in O₂ saturation (<92%) on room air and/or need for supplemental oxygen requirement in order to keep O₂ saturation >92%, (3) need for non-invasive ventilatory support, and (4) critical illness with mechanical respiratory support or evidence for end-organ injury including acute renal failure, heart failure, liver enzyme change or altered mental status.
Time to resolution of COVID-19 Symptoms. COVID-19 symptoms (feverishness, sore throat, cough, shortness of breath, myalgia) will be scored by participants on a 4-point scale (0=none, 1=mild, 2=moderate, 3-severe). Resolution of a symptom is defined as a sustained period (≥7 days) when a symptom previously scored≥1 on the scale was scored as 0).
To evaluate the proportion of patients admitted to hospital for COVID-19 treatment during the 28 days following randomization
To evaluate the change in laboratory measures of inflammation over the 28 days following randomization
To evaluate the safety and tolerability of Mifepristone administered with Amlodipine in addition to SOC as compared to SOC

The primary endpoint is the change from baseline to Day 3 after randomization in nasopharyngeal SARS-CoV-2 viral load (as assessed by quantitative PCR in triplicate) for the two treatment groups

Secondary Endpoints will include:
The change from baseline to Day 5 after randomization in nasopharyngeal SARS-CoV-2 viral load (as assessed by quantitative PCR in triplicate) for the two treatment groups Time to clinical worsening
Time to sustained ≥7 days of clinical resolution
Proportion of patients admitted to hospital during 28 days following randomization
Change in laboratory measures of inflammation over the 28 days following randomization
Percentage of patients with adverse events
Percentage of patients with serious adverse events

Inclusion criteria may include:
Male or females 18 years of age and older
Positive nasopharyngeal SARS-CoV-2 test result by PCR
Symptoms of moderate illness with COVID-19, which could include any symptoms of mild illness or shortness of breath with exertion
Clinical signs suggestive of moderate illness with COVID-19, such as respiratory rate≥20 breaths per minute to maintain a saturation of oxygen (SpO2)<98% but >93% on room air at sea level, heart rate≥90 beats per minute
No clinical signs indicative of severe or critical illness severity or end organ damage
At increased risk of developing severe COVID-19 disease (at least one of the following):
   Age ≥60 years
   Baseline blood pressure under 110 mmHg systolic at rest
   Concomitant need for prescribed medications with known severe interactions with Amlodipine or Mifepristone
   Presence of pulmonary disease including chronic obstructive pulmonary disease, pulmonary hypertension, emphysema
   Diabetes mellitus (type 2), requiring oral medication or insulin for treatment
   Hypertension, requiring at least 1 oral medication for treatment
   History of cardiovascular disease including cardiac and peripheral vascular disease
   Body mass index ≥30

Exclusion criteria may include:
Symptoms of severe systemic illness with COVID-19, which could include any symptom of moderate illness or shortness of breath at rest, or respiratory distress
Clinical signs indicative of severe systemic illness with COVID-19, such as respiratory rate ≥30 per minute, heart rate ≥125 per minute, SpO2≤93% on room air at sea level or PaO2/FiO2<300, and hypotension
Patients who are pregnant, breastfeeding or have a positive pregnancy result before enrollment
Known hypersensitivity to amlodipine, another dihydropyridine or mifepristone
History arrythmia, concurrent use of anti-arrhythmic drugs or family history of sudden cardiac death
Severe liver damage (Child-Pugh score≥C, AST>5 times the upper limit)
Patients with known severe renal impairment (creatinine clearance≤30 mL/min) or patients receiving continuous renal replacement therapy, hemodialysis, or peritoneal dialysis
Receipt of experimental treatment for SARS-CoV-2 (off-label, compassionate use or trial related) within 30 days prior to the time of screening
Asthma requiring glucocorticosteroid treatment
Patient with existing amlodipine or mifepristone exposure within the prior 28 days
Immunocompromised status due to disease (e.g., those living with human immunodeficiency virus with a CD4 T-cell count of <200/mm3)
Immunocompromised status due to medication (e.g., persons taking 20 mg or more of prednisone equivalents a day, anti-inflammatory monoclonal antibody therapies, or cancer therapies)

### Example 2

The antiviral activity of amlodipine and mifepristone, as well as a combination of both, was evaluated against SARS-CoV-2 (strain USA-WA1/2020) in a highly differentiated, three dimensional (3-D), in vitro model of normal human bronchial (dNHBE) cells. The compounds was tested at a single concentrations (0.1 μM for Amlodipine and 5 μM for Mifepristone) in duplicate inserts of 3D tissue models of the human airway (MatTek Corporation). Antiviral activity was measured by virus yield reduction assays 5 days after infection.

Compounds: The test compound was provided as a solid and stored at 4° C. upon arrival. Just before the assay, the compounds were dissolved in 100% DMSO at a 200× concentration. The compounds were further diluted to the test dilutions in the MatTek culture medium (AIR-100-MM).

Cell Culture: Differentiated normal human bronchial epithelial (dNHBE) cells were made to order by MatTek Corporation (Ashland, Mass.) and arrived in kits with either 12- or 24-well inserts each. dNHBE cells were grown on 6mm mesh disks in transwell inserts. During transportation the tissues were stabilized on a sheet of agarose, which was removed upon receipt. One insert was estimated to consist of approximately 1.2×106 cells. Kits of cell inserts (EpiAirway™ AIR-100, AIR-112) originated from a single donor, #9831, a 23-year old, healthy, non-smoking, Caucasian male. The cells have unique properties in forming layers, the apical side of which is exposed only to air and that creates a mucin layer. Upon arrival, the cell transwell inserts were immediately transferred to individual wells of a 6-well plate according to manufacturer's instructions, and 1 mL of MatTek's proprietary culture medium (AIR-100-MM) was added to the basolateral side, whereas the apical side was exposed to a humidified 5% CO2 environment. Cells were cultured at 37° C. for one day before the start of the experiment. After the 24 h equilibration period, the mucin layer, secreted from the apical side of the cells, was removed by washing with 400 μL pre-warmed 30 mM HEPES buffered saline solution 3×. Culture medium was replenished following the wash steps.

Viruses: SARS-CoV-2 strain USA-WA1/2020 was passaged twice in Vero 76 cells to create the virus stock. Virus was diluted in AIR-100-MM medium before infection, yielding a multiplicity of infection (MOI) of approximately 0.003 CCID50 per cell.

Experimental design: Each compound treatment (120 μL) and virus (120 μL) was applied to the apical side, and compound treatment only was applied to the basal side (1 mL), for a 2 h incubation. As a virus control, 3 of the cell wells were treated with placebo (cell culture medium only). Following the 2 h infection, the apical medium was removed, and the basal side was replaced with fresh compound or medium. The cells were maintained at the air liquid interface. On day 5, the medium was removed and discarded from the basal side. Virus released into the apical compartment of the tissues was harvested by the addition of 400 μL of culture medium that was pre-warmed at 37° C. The contents were incubated for 30 min, mixed well, collected, thoroughly vortexed and plated on Vero 76 cells for VYR titration. Triplicate and singlet wells were used for virus control and cell controls, respectively.

Determination of virus titers from each treated cell culture: Vero 76 cells were seeded in 96-well plates and grown overnight (37° C.) to 90% confluence. Samples containing virus were diluted in 10-fold increments in infection medium and 200 μL of each dilution transferred into respective wells of a 96-well microtiter plate. Four microwells were used for each dilution to determine 50% viral endpoints. After 5 days of incubation, each well was scored positive for virus if any cytopathic effect (CPE) was observed as compared with the uninfected control, and counts were confirmed for endpoint on days 6 and 7. The virus dose that was able to infect 50% of the cell cultures (CCID50 per 0.1 mL) was calculated by the Reed-Muench method (1948). The day 5 values are reported. Untreated, uninfected cells were used as the cell controls.

The positive control, remdesivir, was highly active against SARS-CoV-2 in dNHBE cells with an EC90 of 0.009, which is similar to previous results in this assay (Table 1). Since only a single concentration of amlodipine and mifepristone, the EC90 could not be determined. We determined the Log 10 reduction in virus titer (CCID50) by calculating the difference in average Log 10 CCID50 titer of the compound-treated wells and the average titer of the virus control wells. An almost 1 Log 10 reduction in virus titer was observed for both amlodipine and mifepristone, although it did not achieve a full Log 10 reduction (Table 1). The compounds had efficacy at the test concentrations. The combination of both compounds resulted in a similar titer reduction to each compound alone.

TABLE 1

| | | ^{a}Log10 | | | |
|---|---|---|---|---|---|
| Test | Concentration | CCID50 virus | | Log10 | EC90 |
| Compounds | (μM) | per 0.2 mL | Average | Reduction | (μM) |
| Amlodipine | 0.1 | 3.7 | 3.6 | 0.7 | — |
| | | 3.5 | | | |
| Mifepristone | 5 | 3.3 | 3.5 | 0.8 | — |
| | | 3.7 | | | |
| Amlodipine + | 0.1 | 3.3 | 3.4 | 0.9 | — |
| Mifepristone | 5 | 3.5 | | — | |
| Remdesivir | 1.0 | 2.0 | | | 0.009 |
| | 0.1 | 2.7 | | | |
| | 0.01 | 3.3 | | | |
| | 0.001 | 4.0 | | | |
| Virus Control | | 3.7 | 4.3 | | |
| | | 4.5 | | | |
| | | 4.5 | | | |
| | | 4.7 | | | |

Each well was scored positive for virus if any CPE was observed as compared with the uninfected control. Vero 76 cells were scored on day 5 and confirmed on days 6 & 7. ^{a}Titer results from the virus yield reduction assay.^{b}EC90=90% effective concentration (concentration to reduce virus yield by 1 log 10) determined by regression analysis.

Other uses of the disclosed methods will become apparent to those in the art based upon, inter alia, a review of this patent document.

1. A method of treatment of COVID-19 comprising:
administering daily to an individual in need thereof a therapeutically effective dose of mifepristone, or a pharmaceutically acceptable salt and/or analogue thereof, and a therapeutically effective dose of amlodipine, or a pharmaceutically acceptable salt thereof.

2. A method of treatment of COVID-19 hyperinflammation comprising:
administering to an individual in need thereof a therapeutically effective dose of mifepristone, or a pharmaceutically acceptable salt and/or analogue thereof, and a therapeutically effective dose of amlodipine, or a pharmaceutically acceptable salt thereof.

3. A method of reducing, retarding or otherwise inhibiting growth and/or replication of SARS-CoV-2 in an individual confirmed to have COVID-19 comprising:
administering to an individual in need thereof a therapeutically effective dose of mifepristone, or a pharmaceutically acceptable salt and/or analogue thereof, and a therapeutically effective dose of amlodipine, or a pharmaceutically acceptable salt thereof.

4. A method of reducing lung injury resulting from a hyperaggressive immune response comprising:
administering to an individual in need thereof a therapeutically effective dose of mifepristone, or a pharmaceutically acceptable salt and/or analogue thereof, and a therapeutically effective dose of amlodipine, or a pharmaceutically acceptable salt thereof.

5. A method of preventing acute respiratory distress syndrome (ARDS) comprising:
administering to an individual in need thereof a therapeutically effective dose of mifepristone, or a pharmaceutically acceptable salt and/or analogue thereof, and a therapeutically effective dose of amlodipine, or a pharmaceutically acceptable salt thereof.

6. A method of preventing COVID-19 comprising:
administering to an individual at risk of infection with SARS-CoV-2 a therapeutically effective dose of mifepristone, or a pharmaceutically acceptable salt and/or analogue thereof, and a therapeutically effective dose of amlodipine, or a pharmaceutically acceptable salt thereof, prior to exposure to SARS-CoV-2.

7. A method of preventing infection of an individual exposed to SARS-CoV-2 wherein the individual has not been confirmed to have COVID-19 comprising:
administering to the individual a therapeutically effective dose of mifepristone, or a pharmaceutically acceptable salt and/or analogue thereof, and a therapeutically effective dose of amlodipine, or a pharmaceutically acceptable salt thereof.

8. A a method for maintaining vascular endothelial stability, preventing hypovolemic shock and/or improving immune cell viability in an individual infected by coronavirus comprising: administering to the individual a therapeutically effective dose of mifepristone, or a pharmaceutically acceptable salt and/or analogue thereof, and a therapeutically effective dose of amlodipine, or a pharmaceutically acceptable salt thereof.

9. A method for preventing apoptosis of immune cells, particularly macrophage and dendritic cells in an individual infected with SARS-CoV-2, comprising: administering to the individual a therapeutically effective dose of mifepristone, or a pharmaceutically acceptable salt and/or analogue thereof, and a therapeutically effective dose of amlodipine, or a pharmaceutically acceptable salt thereof.

10. A method for preventing vascular endothelial instability, muscle pain, and/or rheumatic pain which an individual may suffer from coronavirus infection comprising:
administering to the individual a therapeutically effective dose of mifepristone, or a pharmaceutically acceptable salt and/or analogue thereof, and a therapeutically effective dose of amlodipine, or a pharmaceutically acceptable salt thereof.

11. The method of any one of the preceding claims, wherein the COVID-19 is of moderate severity.

12. The method of any one of the preceding claims, wherein the administration of mifepristone, or a pharmaceutically acceptable salt and/or analogue thereof, and the amlodipine, or a pharmaceutically acceptable salt thereof, is conducted on an outpatient basis.

13. The method of any one of the preceding claims, wherein the administration of mifepristone, or a pharmaceutically acceptable salt and/or analogue thereof, and the amlodipine, or a pharmaceutically acceptable salt thereof, provides a therapeutically effective plasma concentration or a therapeutically effective concentration at the site of infection of mifepristone, or a pharmaceutically acceptable salt and/or analogue thereof, and a therapeutically effective plasma concentration or a therapeutically effective concentration at the site of infection of the amlodipine, or a pharmaceutically acceptable salt thereof.

14. The method of any one of the preceding claims, wherein the therapeutically effective plasma concentration of mifepristone, or a pharmaceutically acceptable salt and/or analogue thereof is 500 ng/mL to 12,000 ng/mL.

15. The method of any one of the preceding claims, wherein the therapeutically effective plasma concentration of amlodipine, or a pharmaceutically acceptable salt thereof is 0.1 to 10 ng/mL.

16. The method of claim 15, wherein the therapeutically effective plasma concentration of amlodipine, or a pharmaceutically acceptable salt thereof is 0.5 to 10 ng/mL.

17. The method of any one of the preceding claims, wherein the therapeutically effective dose of mifepristone, or a pharmaceutically acceptable salt and/or analogue thereof is at least about 300 mg daily.

18. The method of claim 17, wherein the therapeutically effective dose of mifepristone, or a pharmaceutically acceptable salt and/or analogue thereof is at least about 600 mg daily.

19. The method of claim 17, wherein the therapeutically effective dose of mifepristone, or a pharmaceutically acceptable salt and/or analogue thereof is at least about 1200 mg daily.

20. The method of claim 19, wherein the therapeutically effective dose of mifepristone, or a pharmaceutically acceptable salt and/or analogue thereof is about 1200 mg daily.

21. The method of any one of the preceding claims, wherein the therapeutically effective dose of amlodipine, or a pharmaceutically acceptable salt thereof is at least about 5 mg daily.

22. The method of claim 21, wherein the therapeutically effective dose of amlodipine, or a pharmaceutically acceptable salt thereof is at least about 10 mg daily.

23. The method of claim 22, wherein the therapeutically effective dose of amlodipine, or a pharmaceutically acceptable salt thereof is about 10 mg daily.

24. The method of any one of the preceding claims, wherein the therapeutically effective dose of mifepristone, or a pharmaceutically acceptable salt and/or analogue thereof, and the therapeutically effective dose of amlodipine, or a pharmaceutically acceptable salt thereof is administered for 2 to 14 days.

25. The method of claim 24, wherein the therapeutically effective dose of mifepristone, or a pharmaceutically acceptable salt and/or analogue thereof, and the therapeutically effective dose of amlodipine, or a pharmaceutically acceptable salt thereof is administered for 3 to 12 days.

26. The method of claim 25, wherein the therapeutically effective dose of mifepristone, or a pharmaceutically acceptable salt and/or analogue thereof, and the therapeutically effective dose of amlodipine, or a pharmaceutically acceptable salt thereof is administered for 5 to 10 days.

27. The method of any one of the preceding claims, wherein administration is initiated within the earlier of 24 hours to 72 of illness onset or confirmation of the individual having COVID-19.

28. The method of claim 27, wherein administration is initiated within the earlier of 24 hours of illness onset or confirmation of the individual having COVID-19.

29. The method of any one of the preceding claims, wherein the individual is at an elevated risk of exposure to SARS-CoV-2.

30. The method of claim 29, wherein the individual is a health care worker.

31. The method of claim 29, wherein the individual is located in an area where ongoing community spread of SARS-CoV-2 has been reported.

32. The method of claim 29, wherein the individual has been in close contacts with one or more persons with COVID-19.

33. The method of any one of the preceding claims, wherein the individual is at an elevated risk of severe illness.

34. The method of claim 33, wherein the individual is 60 years of age or older.

35. The method of claim 33, wherein the individual has a serious chronic medical condition.

36. The method of claim 35, wherein the chronic medical condition is chosen from pulmonary disease, diabetes mellitus (type 2), requiring oral medication or insulin for treatment, hypertension, cardiovascular disease.

37. The method of claim 33, wherein the individual has a baseline blood pressure under 110 mmHg systolic at rest.

38. The method of claim 33, wherein the individual has a body mass index≥30.

39. The method of any one of the preceding claims, further comprising testing the individual for SARS-CoV-2 infection.

40. The method of any one of the preceding claims, further comprising monitoring for adverse events during the administration.

41. The method of any one of the preceding claims, wherein the mifepristone, or a pharmaceutically acceptable salt and/or analogue thereof, and the amlodipine, or a pharmaceutically acceptable salt thereof is administered orally.

42. The method of any one of the preceding claims, wherein the mifepristone, or a pharmaceutically acceptable salt and/or analogue thereof, and the amlodipine, or a pharmaceutically acceptable salt thereof, is formulated as a capsule or tablet suitable for oral administration.

43. The method of any one of the preceding claims, further comprising: administering an antiviral drug.

44. The method of claim 43, wherein the antiviral drug is selected from oseltamivir, remdesivir, an interferon, camostat mesylate, nafamostat mesylate, hydroxychloroquine, chloroquine, ivermectin, ribavirin, adefovir, tenofovir, acyclovir, brivudin, cidofovir, fomivirsen, foscarnet, ganciclovir, penciclovir, amantadine, rimantadine and zanamivir.
